(19) United States
(12) Patent Application Publication
(10) Pub. No.: US 2024/0337712 A1
(43) Pub. Date: Oct. 10, 2024

(54) MAGNETIC RESONANCE SYSTEMS AND CIRCUITS

(71) Applicant: WUHAN UNITED IMAGING LIFE SCIENCE INSTRUMENT CO., LTD., Wuhan (CN)

(72) Inventors: Yuman ZHANG, Shanghai (CN); Youlei XU, Shanghai (CN); Jianfan ZHOU, Shanghai (CN); Renkuan ZHAI, Wuhan (CN)

(73) Assignee: WUHAN UNITED IMAGING LIFE SCIENCE INSTRUMENT CO., LTD., Wuhan (CN)

(21) Appl. No.: 18/749,569

(22) Filed: Jun. 20, 2024

Related U.S. Application Data
(63) Continuation of application No. PCT/CN2022/126764, filed on Oct. 21, 2022.

(30) Foreign Application Priority Data
Dec. 30, 2021 (CN) 202111667375.X

(51) Int. Cl. G01R 33/36; G01R 33/341

(52) U.S. Cl. CPC G01R 33/3607; G01R 33/341; G01R 33/3621

(57) ABSTRACT

The present disclosure provides a magnetic resonance system and circuit. The system includes a magnetic resonance transmitter configured to generate one or more radio frequency signals, a magnetic resonance receiver configured to receive one or more magnetic resonance signals collected by one or more coils, and a magnetic resonance radio frequency power transceiver including a communication circuit and a control circuit. The communication circuit provides a first communication circuit and a second communication circuit. The first communication circuit is configured to transmit the one or more radio frequency signals to the one or more coils. The second communication circuit is configured to transmit the one or more magnetic resonance signals to the magnetic resonance receiver. The control circuit is configured to send one or more control signals to the communication circuit to control the first communication circuit or the second communication circuit to turn on or cut off.

### CROSS-REFERENCE RELATED TO APPLICATIONS

This application is a continuation of international application No. PCT/CN2022/126764 filed on Oct. 21, 2022, which claims priority to Chinese application No. 202111667375.X filed on Dec. 30, 2021, the entire contents of each of which are incorporated herein by reference.

### TECHNICAL FIELD

The present disclosure relates to the field of magnetic resonance, and in particular to magnetic resonance systems and magnetic resonance circuits.

### BACKGROUND

With the enhancement of magnetic resonance field strength, the optimization of coil, and the emergence of ultra-high-speed sequences, the research on magnetic resonance of other nuclides (e.g., hydrogen (1H), carbon (13C), nitrogen (15N), fluorine (19F), sodium (23Na), phosphorus (31P), xenon (129Xe), etc.) has also developed rapidly. For the detection of these nuclides, more comprehensive imaging and evaluation of tissues and organs (e.g., the brain, the heart, the lungs, and other tissues and organs) can be performed. To realize the transmission and reception of radio frequencies of different nuclides, the circuits for transmitting and receiving different nuclides in the magnetic resonance radio frequency power transceiver are not the same. However, the method for switching the circuits through a hardware switch to respectively realize functions of the transmission and reception increases the hardware cost of the magnetic resonance radio frequency transceiver, and the efficiency of the transmission and reception of the radio frequency is relatively low.

Accordingly, it is desirable to provide magnetic resonance systems and magnetic resonance circuits that can effectively reduce the hardware cost and improve the efficiency of the transmission and reception of the radio frequency.

### SUMMARY

According to one of the embodiments of the present disclosure, a magnetic resonance system is provided. The magnetic resonance system includes: a magnetic resonance transmitter configured to generate one or more radio frequency signals; a magnetic resonance receiver configured to receive one or more magnetic resonance signals collected by one or more coils; and a magnetic resonance radio frequency power transceiver, including a communication circuit and a control circuit, wherein the communication circuit provides a first communication circuit and a second communication circuit, the first communication circuit is configured to transmit the one or more radio frequency signals to the one or more coils, and the second communication circuit is configured to transmit the one or more magnetic resonance signals to the magnetic resonance receiver; and the control circuit is configured to send one or more control signals to the communication circuit to control the first communication circuit or the second communication circuit to turn on or cut off.

According to one embodiment of the present disclosure, a magnetic resonance circuit applied to the magnetic resonance system is provided, and the magnetic resonance system further includes a magnetic resonance transmitter and a magnetic resonance receiver. The magnetic resonance circuit includes a communication circuit and a control circuit, wherein: the communication circuit provides the first communication circuit and the second communication circuit, the first communication circuit is configured to transmit the one or more radio frequency signals to the one or more coils, and the second communication circuit is configured to transmit the one or more magnetic resonance signals to the magnetic resonance receiver; and the control circuit is configured to send one or more control signals to the communication circuit to control the first communication circuit or the second communication circuit to turn on or cut off.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating a structure of a magnetic resonance system according to some embodiments of the present disclosure;

FIG. 2 is a schematic diagram illustrating a structure of a magnetic resonance circuit according to some embodiments of the present disclosure;

FIG. 3 is a schematic diagram illustrating a structure of a magnetic resonance circuit according to some embodiments of the present disclosure;

FIG. 4 is a schematic diagram illustrating a structure of a magnetic resonance circuit according to some embodiments of the present disclosure;

FIG. 5 is a schematic diagram illustrating a structure of a magnetic resonance circuit according to some embodiments of the present disclosure;

FIG. 6 is a schematic diagram illustrating a structure of a magnetic resonance circuit according to some embodiments of the present disclosure;

FIG. 7 is a schematic diagram illustrating a structure of a magnetic resonance circuit according to some embodiments of the present disclosure;

FIG. 8 is a schematic diagram illustrating a structure of a magnetic resonance circuit according to some embodiments of the present disclosure;

FIG. 9 is a schematic diagram illustrating a structure of a magnetic resonance system according to some embodiments of the present disclosure; and

FIG. 10 is a schematic diagram illustrating a structure of a magnetic resonance system according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, the drawings described below are only some examples or embodiments of the present disclosure. Those skilled in the art, without further creative efforts, may apply the present disclosure to other similar scenarios according to these drawings. Unless apparent from the locale or otherwise stated, like reference numerals represent similar structures or operations throughout the several views of the drawings.

It will be understood that the term “system,” “device,” “unit,” and/or “module” used herein are one method to distinguish different components, elements, parts, sections, or assembly of different levels in ascending order. However, the terms may be displaced by another expression if they achieve the same purpose.

As used in the disclosure and the appended claims, the singular forms “a,” “an,” and/or “the” may include plural forms unless the content clearly indicates otherwise. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

The embodiments of the present disclosure provide a magnetic resonance system and a magnetic resonance circuit that can be applied to a magnetic resonance system supporting a plurality of nuclides, thereby realizing transmission and reception of the plurality of nuclides in a high field (e.g., magnetic field strength of 5T and above) magnetic resonance broadband. In some embodiments, the magnetic resonance system and the magnetic resonance circuit can be applied to a medical imaging system, a life science instrumentation, and any other system that uses magnetic resonance technology. In some embodiments, the medical imaging system may include a magnetic resonance imaging (MRI) system, a computed tomography-magnetic resonance imaging (MRI-CT) system, a positron emission tomography-magnetic resonance imaging (PET-MRI) system, a single photon emission computed tomography-magnetic resonance imaging (SPECT-MRI) system, a digital subtraction angiography-magnetic resonance imaging (DSA-MRI) system, etc.

In some embodiments, the magnetic resonance system includes a magnetic resonance transmitter, a magnetic resonance receiver, and a magnetic resonance radio frequency power transceiver. The magnetic resonance radio frequency power transceiver includes a communication circuit and a control circuit. The communication circuit provides a first communication circuit and a second communication circuit. The first communication circuit is configured to transmit one or more radio frequency signals to one or more coils. The second communication circuit is configured to transmit one or more magnetic resonance signals to the magnetic resonance receiver. The control circuit is configured to send one or more control signals to the communication circuit to control the first communication circuit or the second communication circuit to turn or cut off. The one or more radio frequency signals may be a plurality of radio frequency signals corresponding to different kinds of nuclides, and the one or more magnetic resonance signals may be a plurality of magnetic resonance signals transmitted by different kinds of nuclides. Compared to providing an independent transmission channel for the radio frequency signal or the magnetic resonance signal corresponding to each kind of nuclides, by adopting a magnetic resonance radio frequency power transceiver supporting broadband connectivity at multiple frequency points, a count of transmission channels between the magnetic resonance transmitter and the coil, as well as between the coil and the magnetic resonance receiver may be effectively reduced, thereby reducing the volume and the hardware cost of the magnetic resonance system, and improving operating efficiency of transmitting and receiving the radio frequency.

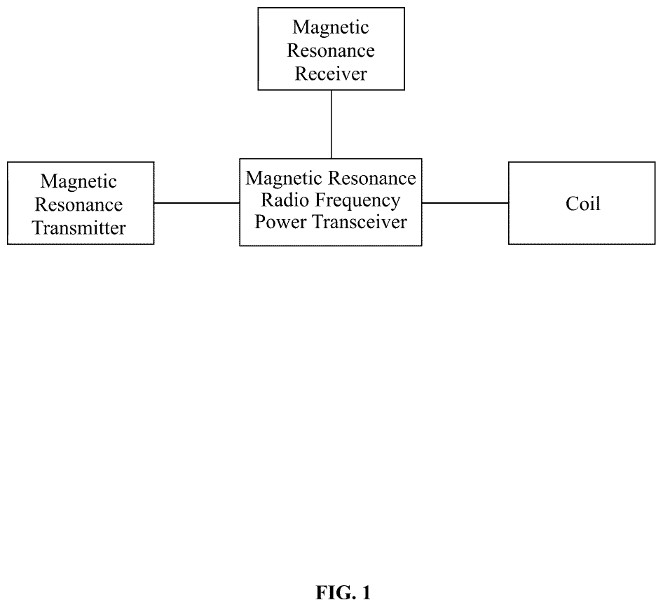

FIG. 1 is a schematic diagram illustrating a structure of a magnetic resonance system according to some embodiments of the present disclosure.

As shown in FIG. 1, the magnetic resonance system may include a magnetic resonance transmitter, a magnetic resonance receiver, a coil, and a magnetic resonance radio frequency power transceiver.

The magnetic resonance transmitter is connected to the magnetic resonance radio frequency power transceiver to generate one or more radio frequency signals at different moments or the same moment. The one or more radio frequency signals may be a plurality of radio frequency signals corresponding to different kinds of nuclides. In the magnetic resonance imaging technique, a radio frequency transmitting coil may be utilized to send a radio frequency signal to a detection object, thereby exciting a specific frequency of one or more nuclides in the detection object to resonate to generate one or more magnetic resonance signals. Since resonance frequencies of different kinds of nuclides are different, it is necessary to use different frequencies of radio frequency signals to excite different kinds of nuclides to generate different magnetic resonance signals. The detection of the relevant nuclides may be used to perform more comprehensive imaging and assessment on tissues and organs (e.g., the brain, the heart, the lungs, etc.). In some embodiments, different kinds of nuclides may include hydrogen (1H), carbon (13C), nitrogen (15N), fluorine (19F), sodium (23Na), phosphorus (31P), xenon (129Xe), etc.

The magnetic resonance radio frequency power transceiver is respectively connected to the magnetic resonance transmitter, the magnetic resonance receiver, and the coil to be configured to control the transmission and reception of the radio frequency of the magnetic resonance system. The magnetic resonance radio frequency power transceiver includes a transmitting state and a receiving state. When the magnetic resonance radio frequency power transceiver is in the transmitting state, a transmission channel between the magnetic resonance transmitter and the coil is turned on, and the transmission channel between the coil and the magnetic resonance receiver is cut off. When the magnetic resonance radio frequency power transceiver is in the receiving state, the transmission channel between the coil and the magnetic resonance receiver is turned on, and the transmission channel between the magnetic resonance transmitter and the coil is cut off.

The coil is connected to the magnetic resonance radio frequency power transceiver and configured to generate a magnetic field that excites resonance of different kinds of nuclides or collect one or more magnetic resonance signals, wherein the one or more magnetic resonance signals may be a plurality of magnetic resonance signals transmitted by different kinds of nuclides. In some embodiments, when the magnetic resonance radio frequency power transceiver is in the transmitting state, the transmission channel between the magnetic resonance transmitter and the coil is turned on, at which time the plurality of radio frequency signals corresponding to the different kinds of nuclides generated by the magnetic resonance transmitter is transmitted to the coil through the transmission channel, and the coil generates a magnetic field that excites resonance of the different kinds of nuclides, causing corresponding different kinds of nuclides to generate magnetic resonance signals under an action of the magnetic field. In some embodiments, the magnetic field that excites the resonance of the different kinds of nuclides may include a circularly polarized magnetic field generated by orthogonal excitation. In some embodiments, when the magnetic resonance radio frequency power transceiver is in the receiving state, the transmission channel between the coil and the magnetic resonance receiver is turned on, at which time the coil is used to collect the plurality of magnetic resonance signals emitted by the different kinds of nuclides. In some embodiments, the coil may be provided inside the magnetic resonance system or may be connected to the magnetic resonance radio frequency power transceiver inside the magnetic resonance system as an external component. In some embodiments, the coil may include multiple groups of coils, and each group of coils corresponds to the radio frequency signals or the magnetic resonance signals of the different kinds of nuclides, i.e., each group of coils is respectively used to emit (receive) the radio frequency signals (magnetic resonance signals) of a particular kind of nuclide.

The magnetic resonance receiver is connected to the magnetic resonance radio frequency power transceiver to receive one or more magnetic resonance signals collected by the coil at different moments or the same moment. In some embodiments, when the magnetic resonance radio frequency power transceiver is in the receiving state, the transmission channel between the coil and the magnetic resonance receiver is turned on, and the magnetic resonance signals collected by the coil are transmitted to the magnetic resonance receiver through the transmission channel, which may be imaged after subsequent processing.

In some embodiments, the magnetic resonance radio frequency power transceiver may switch the transmitting state or the receiving state based on a control signal generated by the magnetic resonance system. Merely by way of example, the magnetic resonance radio frequency power transceiver may be set to the transmitting state based on the control signal, i.e., to enter a first operating mode in which the magnetic resonance system may transmit the radio frequency signals generated by the magnetic resonance transmitter to the coil. The magnetic resonance radio frequency power transceiver may be set to the receiving state according to the control signal, i.e., to enter a second operating mode in which the magnetic resonance system may transmit the magnetic resonance signals collected by the coil to the magnetic resonance receiver.

In some embodiments, the magnetic resonance radio frequency power transceiver may provide the same transmission channel for the plurality of radio frequency signals corresponding to the different kinds of nuclides. For example, the plurality of radio frequency signals corresponding to the different kinds of nuclides generated by the magnetic resonance transmitter may be transmitted to the coil through the same transmission channel provided by the magnetic resonance radio frequency power transceiver. In this case, the transmission of the plurality of radio frequency signals corresponding to the different kinds of nuclides may be realized by merely setting a switch for the same transmission channel, without setting a switch for a specific frequency or broadband corresponding to each kind of nuclides, thereby effectively reducing redundant hardware switches. In some embodiments, the magnetic resonance transmitter may simultaneously generate radio frequency signals corresponding to the same kind of nuclide, or the magnetic resonance transmitter may generate a plurality of radio frequency signals corresponding to different kinds of nuclides at different times, and then the radio frequency signals may be transmitted by the same radio frequency power transceiver.

In some embodiments, the magnetic resonance radio frequency power transceiver may enter the first operating mode or the second operating mode according to a control signal, thereby realizing the switching between the transmission channel between the magnetic resonance transmitter and the coil and the transmission channel between the coil and the magnetic resonance receiver transmission channel, without providing different transmission channels for the excitation (or reception) of different nuclides, avoiding the use of excessive hardware switches, and solving a problem of excessive hardware switches, relatively high cost, and relatively low efficiency of the transmission and reception of the radio frequency of the magnetic resonance receiver in the related technology, reducing the volume and the hardware cost of the magnetic resonance device, and improving operating efficiency.

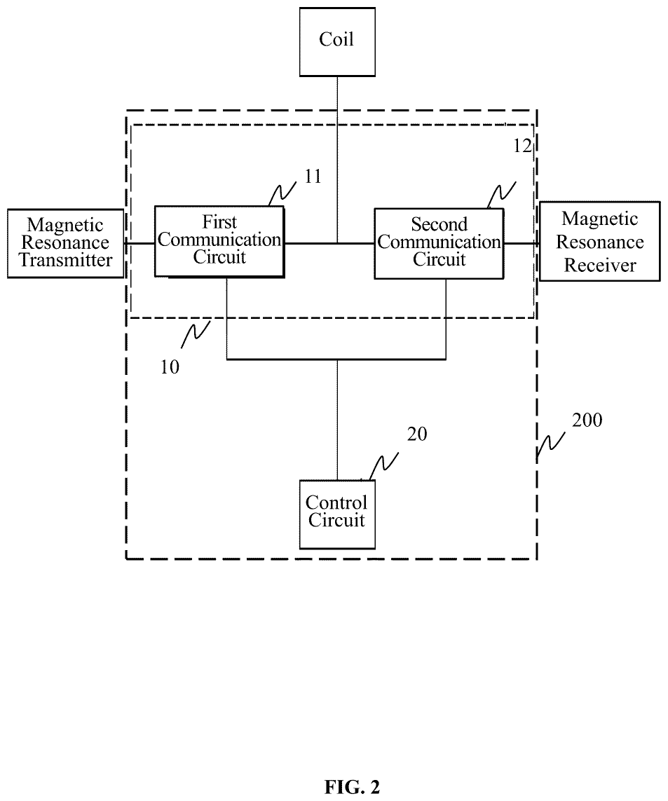

FIG. 2 is a schematic diagram illustrating a structure of a magnetic resonance circuit according to some embodiments of the present disclosure.

As shown in FIG. 2, a magnetic resonance circuit 200 is a magnetic resonance radio frequency power transceiver. The magnetic resonance circuit 200 may be applied to a magnetic resonance system including a magnetic resonance transmitter, a magnetic resonance receiver, and a coil. The magnetic resonance circuit 200 may include a communication circuit 10 and a control circuit 20. The communication circuit 10 includes a first communication circuit 11 and a second communication circuit 12. The first communication circuit 11 is used to transmit one or more radio frequency signals to the coil, the second communication circuit 12 is used to transmit one or more magnetic resonance signals to the magnetic resonance receiver, and the control circuit 20 is used to send a control signal to control the first communication circuit 11 or the second communication circuit 12 to turn on or cut off. The control signal includes a turn-on signal and/or a cut-off signal. Specifically, the first communication circuit 11 may be used to transmit a plurality of radio frequency signals corresponding to different kinds of nuclides to the coil, and the second communication circuit 12 is used to transmit a plurality of magnetic resonance signals emit by the different kinds of nuclides to the magnetic resonance receiver. In some embodiments, the control circuit 20 may include a power supply, and the power supply is used to output the turn-on signal and/or the cut-off signal.

As shown in FIG. 2, one end of the first communication circuit 11 is connected to the magnetic resonance transmitter, and another end of the first communication circuit 11 is connected to the second communication circuit 12. One end of the second communication circuit 12 is connected to the magnetic resonance receiver. The coil is connected to one end where the first communication circuit 11 and the second communication circuit 12 are connected, and the control circuit 20 is connected to the first communication circuit 11 and the second communication circuit 12, respectively.

In some embodiments, the control circuit 20 may receive a system instruction of the magnetic resonance system, generate a control signal based on the system instruction, and send the control signal to the communication circuit 10 to control the first communication circuit 11 in the communication circuit 10 to turn on or cut off, as well as the second communication circuit 12 to turn on or cut off. Specifically, the first communication circuit 11 is used to connect the magnetic resonance transmitter and the coil to send the radio frequency signals output from the magnetic resonance transmitter to the coil, and the second communication circuit 12 is used to connect the coil and the magnetic resonance receiver to send the magnetic resonance signals collected by the coil to the magnetic resonance receiver. In some embodiments, the system instruction may be automatically generated by the magnetic resonance system or manually input by a user, and the user may control the communication circuit 10 to turn on or cut off through the system instruction.

In some embodiments, the first communication circuit 11 and the second communication circuit 12 may be provided with a first switch and a second switch, respectively. The closure of the first switch or closure of the second switch is realized through the control signal sent by the control circuit 20, thereby realizing the turn-on of the first communication circuit 11 or the turn-on of the second communication circuit 12. In some embodiments, the communication circuit 10 may be provided with a dual-channel selection switch. The dual-channel selection switch is connected to the first communication circuit 11 and the second communication circuit 12, respectively. The closure of one channel of the dual-channel selection switch and the cut off of another channel of the dual-channel selection switch are realized by the control signal sent by the control circuit 20, thereby realizing the turn-on of the first communication circuit 11 or the turn-on of the second communication circuit 12.

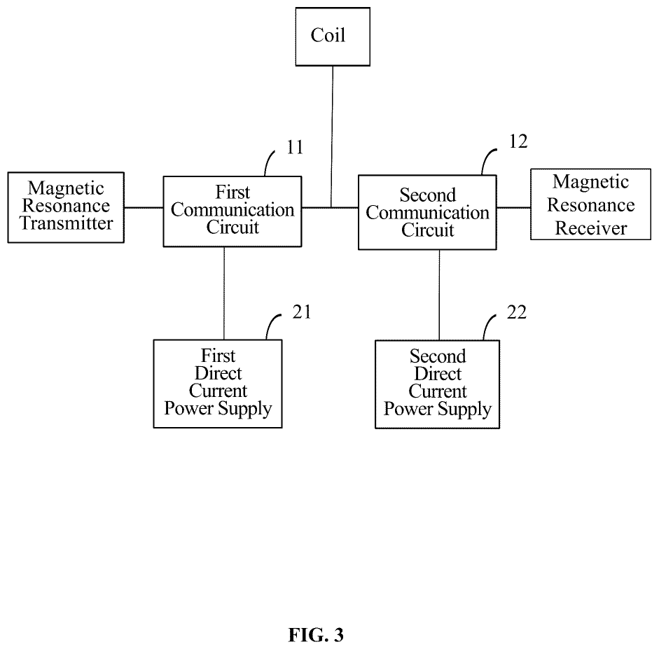

FIG. 3 is a schematic diagram illustrating a structure of a magnetic resonance circuit according to some embodiments of the present disclosure.

As shown in FIG. 3, in some embodiments, the control circuit 20 may include a first direct current power supply 21 and a second direct current power supply 22. The first direct current power supply 21 is connected to the first communication circuit 11, and the second direct current power supply 22 is connected to the second communication circuit 12. The first direct current power supply 21 is used to output a first turn-on signal and/or a first cut-off signal, and the second direct current power supply 22 is used to output a second cut-off signal and/or a second turn-on signal to control the first communication circuit 11 or the second communication circuit 12 to turn on or cut off. In some embodiments, when the magnetic resonance radio frequency power transceiver is required to be set to a transmitting state, the first direct current power supply 21 may output the first turn-on signal and the second direct current power supply 22 may output the second cut-off signal, at this time, the control circuit 20 may control the first communication circuit 11 to turn on and control the second communication circuit 12 to cut off. In some embodiments, when the magnetic resonance radio frequency power transceiver is required to be set to a receiving state, the first direct current power supply 21 may output the first cut-off signal, and the second direct current power supply 22 may output the second direct communication signal, at this time, the control circuit 20 may control the first communication circuit 11 to cut off and control the second communication circuit 12 to turn on.

In some embodiments, the control signal output by the control circuit 20 may include a first control signal and a second control signal. The first control signal may be a superposition signal of the first turn-on signal and the second cut-off signal to cause the first communication circuit 11 to turn on and the second communication circuit 12 to cut off. At this time, the communication circuit 10 is used to transmit the radio frequency signals sent by the magnetic resonance transmitter to the coil. The second control signal may be a superposition signal of the first cut-off signal and the second turn-on signal to cause the first communication circuit 11 to turn off and the second communication circuit 12 to turn on. At this time, the communication circuit 10 is used to transmit the magnetic resonance signals collected by the coil to the magnetic resonance receiver.

In some embodiments, by controlling the turn on and cut off of the first communication circuit 11 and the second communication circuit 12 by the first direct current power supply 21 and the second direct current power supply 22, respectively, there is no need for a complicated circuit control structure, and the switching of the communication circuit 10 can be realized by only switching the turn-on signal and the cut-off signal output from the first direct current power supply 21 and the second direct current power supply 22, thereby further realizing the switching between the transmission channel between the magnetic resonance transmitter and the coil and the transmission channel between the coil and the magnetic resonance receiver, and the process of realizing the switching is simple and the hardware cost of the control circuit 20 is reduced.

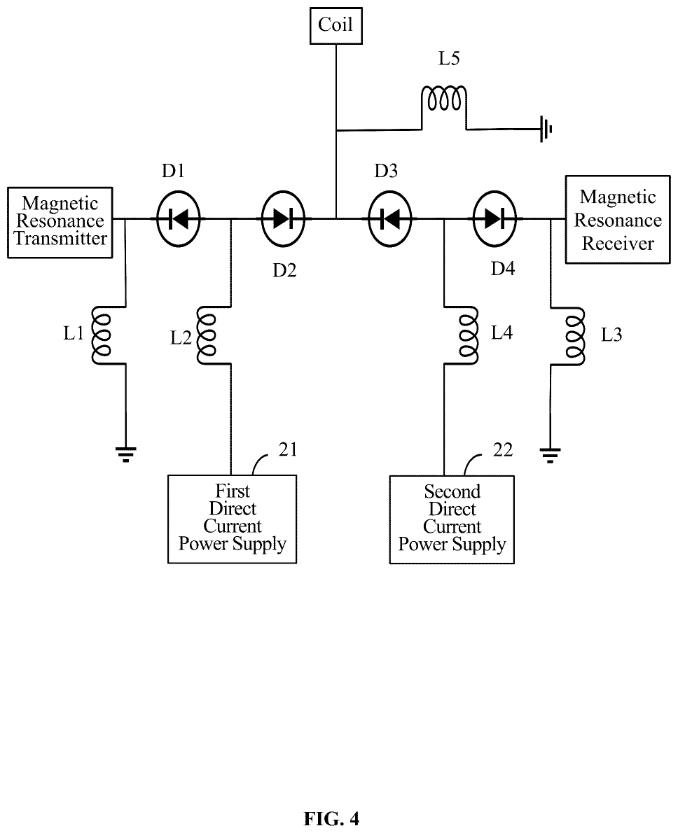

FIG. 4 is a schematic diagram illustrating a structure of a magnetic resonance circuit according to some embodiments of the present disclosure.

As shown in FIG. 4, in some embodiments, the first communication circuit 11 may include a first diode D1 and a second diode D2, and the second communication circuit 12 includes a third diode D3 and a fourth diode D4. One end (the negative electrode) of the first diode D1 is connected to the magnetic resonance transmitter, one end (the positive electrode) of the second diode D2 is connected to another end (the positive electrode) of the first diode D1, and another end (the negative electrode) of the second diode D2 is connected to the coil. One end (the negative electrode) of the third diode D3 is connected to the coil, one end (the positive electrode) of the fourth diode D4 is connected to another end (the positive electrode) of the third diode D3, and another end (the negative electrode) of the fourth diode D4 is connected to the magnetic resonance receiver. The diode refers to a diode with a switching function, for example, a photodiode. In some embodiments, the diode may include one or more of a pass-switching diode, a high-speed switching diode, an ultra-high-speed switching diode, a low-power switching diode, a high-reverse-voltage switching diode, a silicon voltage switching diode, etc.

In some embodiments, in order to meet power requirements of the communication circuit 10 for exciting different nuclides, one or more of the diodes (e.g., the first diode D1, the second diode D2, the third diode D3, and the fourth diode D4) need to be able to withstand a certain voltage and a certain current. Merely by way of example, the first diode D1 may withstand a maximum high voltage of 3000V and a current of 25 A, and a minimum voltage of 230V and a current of 8 A. Optionally, in order to be suitable for the magnetic resonance environment of magnetic resonance system applications, the diodes in the communication circuit 10 are non-magnetic structures.

Merely by way of example, the diode is a switching diode, and the first diode D1, the second diode D2, the third diode D3, and the fourth diode D4 are provided in the magnetic resonance circuit based on the characteristics of forward-biased conduction and reverse-biased cutoff of the switching diode. Specifically, the first diode D1 and the second diode D2 form the first communication circuit 11. When the first direct current power supply 21 inputs the first turn-on signal, the first diode D1 and the second diode D2 are in a turn-on state; and when the first direct current power supply 21 inputs the first cut-off signal, the first diode D1 and the second diode D2 are in a cut-off state. The third diode D3 and the fourth diode D4 form the second communication circuit 12. When the second direct current power supply 22 inputs the second turn-on signal, the third diode D3 and the fourth diode D4 are in the turn-on state; and when the second direct current power supply 22 inputs the second cut-off signal, the third diode D3 and the fourth diode D4 are in the cut-off state.

In some embodiments, the first turn-on signal and the second turn-on signal are positive voltages that exceed a turn-on voltage of the switching diode. The first cut-off signal and the second cut-off signal are negative voltages below a cut-off voltage of the diode. The numerical magnitudes of the first cut-off signal and the second cut-off signal may be set based on a radio frequency power amplifier in the magnetic resonance system. It should be appreciated that the first turn-on signal and the second turn-on signal in this embodiment may be set to exceed the turn-on voltage of the switching diode to drive the switching diode to turn on. The first cut-off signal and the second cut-off signal may be set to be lower than the cut-off voltage of the switching diode to cut off the switching diode. In some embodiments, the first turn-on signal is a positive voltage that exceeds the turn-on voltage of the first diode D1 and the turn-on voltage of the second diode D2, and the second turn-on signal is a positive voltage that exceeds the turn-on voltage of the third diode D3 and the turn-on voltage of the fourth diode D4. The first cut-off signal is a negative voltage below the cut-off voltage of the first diode D1 and the cut-off voltage of the second diode D2, and the second cut-off signal is a negative voltage below the cut-off voltage of the third diode D3 and the cut-off voltage of the fourth diode D4.

Merely by way of example, the first direct current power supply 21 inputs the first turn-on signal and the second direct current power supply 22 inputs the second cut-off signal, at which time the first diode D1 and the second diode D2 are in the turn-on state and the third diode D3 and the fourth diode D4 are in the cut-off state, i.e., the first communication circuit 11 is turned on, and the second communication circuit 12 is cut off. At this time, the magnetic resonance system can be used to transmit the radio frequency signals sent by the magnetic resonance transmitter to the coil to further generate the magnetic resonance signals. The first direct current power supply 21 inputs the first cut-off signal, and the second direct current power supply 22 inputs the second turn-on signal, at which time the first diode D1 and the second diode D2 are in the cut-off state, and the third diode D3 and the fourth diode D4 are in the turn-on state, i.e., the first communication circuit 11 is cut off, and the second communication circuit 12 is turned on. At this time, the magnetic resonance system can be used to transmit the magnetic resonance signals collected by the coil to the magnetic resonance receiver for subsequent processing.

In some embodiments, based on the property that an inductor passes direct current and blocks alternating current, the magnetic resonance circuit may further include an inductor element. The inductor element is connected among the magnetic resonance transmitter, the coil, the magnetic resonance receiver, the communication circuit, and the control circuit. In some embodiments, the inductor element includes a first inductor L1, a third inductor L3, and a fifth inductor L5. One end of the first inductor L1 is connected to the magnetic resonance transmitter, and another end of the first inductor L1 is grounded. One end of the third inductor L3 is connected to the magnetic resonance receiver, and another end of the third inductor L3 is grounded. One end of the fifth inductor L5 is connected to the coil, and another end of the fifth inductor L5 is grounded. In some embodiments, the inductor element further includes a second inductor L2 and a fourth inductor L4. The second inductor L2 is connected between the first direct current power supply 21 and the first communication circuit 11, and the fourth inductor L4 is connected between the second direct current power supply 22 and the second communication circuit 12. Specifically, one end of the second inductor L2 is connected to the first direct current power supply 21, and another end of the second inductor L2 is respectively connected to the positive electrode of the first diode D1 and the positive electrode of the second diode D2. One end of the fourth inductor L4 is connected to the second direct current power supply 22, and another end of the fourth inductor L4 is respectively connected to the positive electrode of the third diode D3 and the positive electrode of the fourth diode D4.

The first inductor L1 and the second inductor L2 may be used to isolate the radio frequency signals output by the magnetic resonance transmitter, and may also be used to conduct the direct current output by the first direct current power supply 21. The third inductor L3 and the fourth inductor L4 may be used to isolate collected signals output by the coil, and may also be used to conduct the direct current output by the second direct current power supply 22. The fifth inductor L5 may be used to isolate the radio frequency signals output by the magnetic resonance transmitter and the magnetic resonance signals output by the coil, and may also be used to conduct the direct current output by the first direct current power supply 21 and the direct current output by the second direct current power supply 22.

In some embodiments, when the first communication circuit 11 is conducted, since the first direct current power supply 21 outputs the direct current and the radio frequency signal output by the magnetic resonance transmitter is an alternating current signal, the first inductor L1, the second inductor L2, and the fifth inductor L5 conduct the direct current output by the first direct current power supply 21 and isolate the transmission signal sent by the magnetic resonance transmitter to the coil, thereby avoiding loss of the transmission signal during transmission. When the second communication circuit 12 is conducted, since the second direct current power supply 22 outputs the direct current and the magnetic resonance signal collected by the coil is the alternating current signal, the third inductor L3, the fourth inductor L4, and the fifth inductor L5 conduct the direct current output by the second direct current power supply 22 and isolate the collected signal sent by the coil to the magnetic resonance receiver, thereby avoiding the loss of the collected signal during transmission.

In the embodiments of the present disclosure, the communication circuit 10 is formed by the first diode D1, the second diode D2, the third diode D3, and the fourth diode D4, such that the switching between the conduction of the first communication circuit 11 and the conduction of the second communication circuit 12 is realized according to the turn-on signals or the cut-off signals of the direct current power supply 21 and the second direct current power supply 22. The above scheme is easy to implement without manual operation. Further, the first inductor L1, the second inductor L2, the third inductor L3, the fourth inductor L4, and the fifth inductor L5 are disposed in the magnetic resonance circuit to isolate the radio frequency signals and the collected magnetic resonance signals during transmission, which avoids the loss of the radio frequency signals and the collected magnetic resonance signals during transmission to ensure signal quality, thereby improving the detection accuracy of the magnetic resonance system.

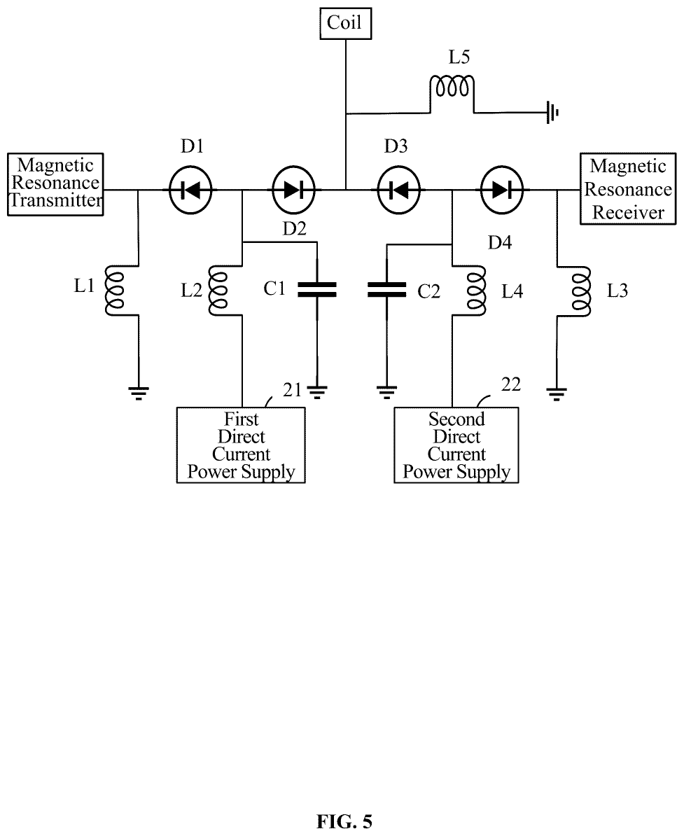

FIG. 5 is a schematic diagram illustrating a structure of a magnetic resonance circuit according to some embodiments of the present disclosure.

As shown in FIG. 5, the magnetic resonance circuit may further include a capacitive element. The capacitive element, together with the second inductor L2 and the fourth inductor L4, may form a filter circuit for filtering out interferences from signals other than the control signal, the plurality of radio frequency signals, and the plurality of magnetic resonance signals. In some embodiments, the capacitive element may include a first capacitor C1 and a second capacitor C2. One end of the first capacitor C1 is connected to the second inductor L2, and another end of the first capacitor C1 is grounded. One end of the second capacitor C2 is connected to the fourth inductor L4, and another end of the second capacitor C2 is grounded.

The first capacitor C1 and the second inductor L2 form a first filter circuit. The second capacitor C2 and the fourth capacitor L4 form a second filter circuit. In some embodiments, to meet the requirements of the magnetic resonance circuit for filtering out interferences from other signals, and to realize the transmission and reception of multiple kinds of nucides under an ultra-high field magnetic resonance broadband, the capacitive element (e.g., the first capacitor C1 and the second capacitor C2) needs to have a high voltage resistance and a good radio frequency characteristic. In some embodiments, the capacitive element may be an RF high-power capacitor.

In some embodiments, the first filter circuit composed of the first capacitor C1 and the second inductor L2 is a low-pass filter circuit, and the first filter circuit may be used to filter out the interferences from other signals to ensure that the control signal of the first direct current power supply can be transmitted to the diode through the filter circuit, and at the same time to ensure that the radio frequency signals generated by the magnetic resonance transmitter can arrive at the coil completely.

In some embodiments, the second filter circuit formed by the second capacitor C2 and the fourth inductor L4 is also a low-pass filter circuit, and the second filter circuit may be used to filter out the interferences from other signals to ensure that the control signal of the second direct current power supply can be transmitted to the diode through the filter circuit, and at the same time to ensure that the magnetic resonance signal collected by the coil can arrive at the magnetic resonance receiver completely.

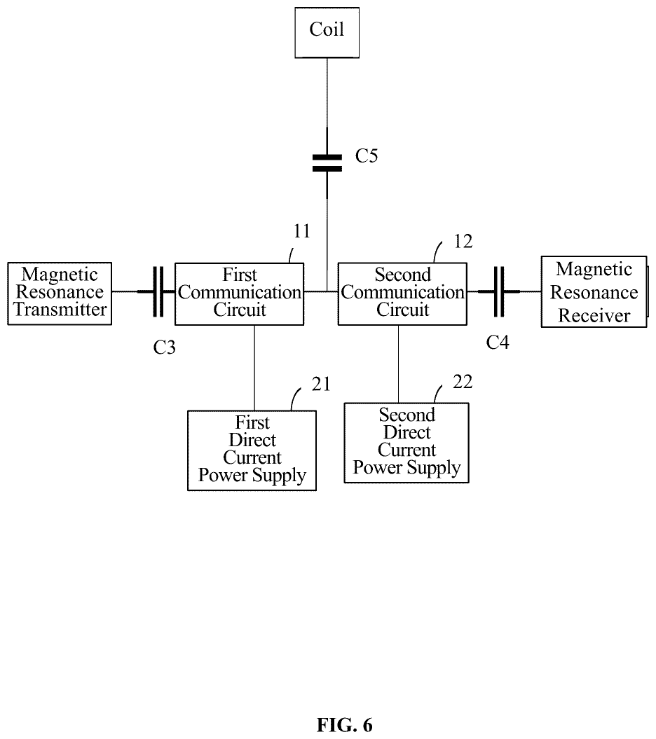

FIG. 6 is a schematic diagram illustrating a structure of a magnetic resonance circuit according to some embodiments of the present disclosure.

As shown in FIG. 6, in some embodiments, based on the property that a capacitor passes the alternating current and blocks the direct current, the magnetic resonance circuit may include a third capacitor C3, a fourth capacitor C4, and a fifth capacitor C5. One end of the third capacitor C3 is connected to the magnetic resonance transmitter, and another end of the third capacitor C3 is connected to the first communication circuit 11. One end of the fourth capacitor C4 is connected to the magnetic resonance receiver, and another end of the fourth capacitor C4 is connected to the second communication circuit 12. One end of the fifth capacitor C5 is connected to the coil, and another end of the fifth capacitor C5 is respectively connected to the first communication circuit 11 and the second communication circuit 12. The third capacitor C3 may be used to isolate the direct current output by the first direct current power supply 21, the fourth capacitor C4 may be used to isolate the direct current output by the second direct current power supply 22, and the fifth capacitor C5 may be used to isolate the direct current output by the first direct current power supply 21 and the direct current output by the second direct current power supply 22. In some embodiments, the third capacitor C3, the fourth capacitor C4, or the fifth capacitor C5 may also be an RF high-power capacitor.

In some embodiments, when the first communication circuit 11 is conducted, the third capacitor C3 may be used to isolate a current output by the first direct current power supply 21 to avoid the current passing through the magnetic resonance transmitter. When the second communication circuit 12 is conducted, the fourth capacitor C4 may be used to isolate the current output from the second direct current power supply 22 to avoid the current passing through the magnetic resonance receiver. The fifth capacitor C5 may be used to isolate the current output from the first direct current power supply 21 and the current output from the second direct current power supply 22 to avoid the currents output from the first direct current power supply 21 and the second direct current power supply 22 passing through the coil.

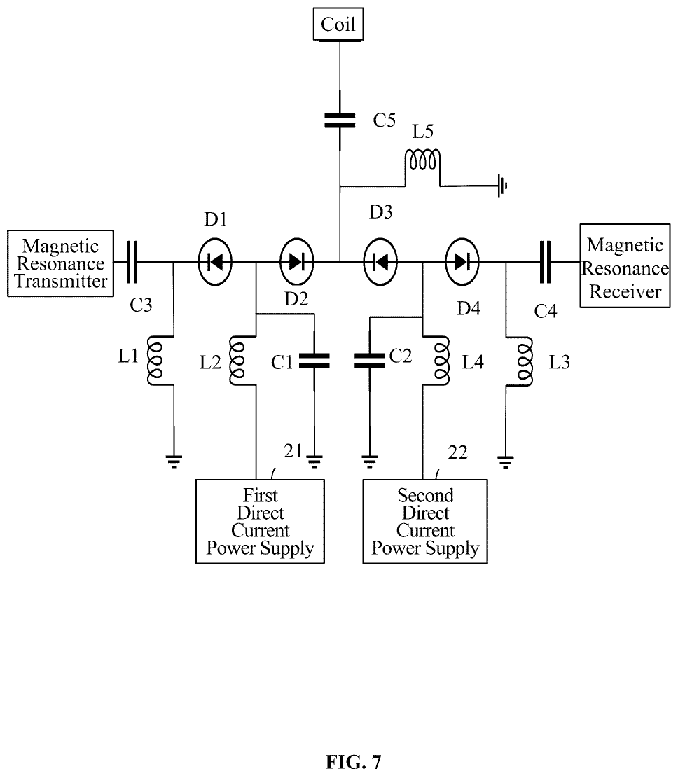

FIG. 7 is a schematic diagram illustrating a structure of a magnetic resonance circuit according to some embodiments of the present disclosure.

As shown in FIG. 7, in some embodiments, when the magnetic resonance transmitter needs to transmit a radio frequency signal to the coil, the first direct current power supply 21 outputs the first turn-on signal. The first diode D1 is conducted along a direction of the second inductor L2, the first diode D1, and the first inductor L1. The second diode D2 is conducted along a direction of the second inductor L2, the second diode D2, and the fifth inductor L5. At this time, the transmission channel from the magnetic resonance transmitter to the coil is opened.

At the same time, the second direct current power supply 22 outputs the second cut-off signal, causing the third diode D3 and the fourth diode D4 to be in a cut-off state, i.e., the coil to the magnetic resonance receiver is in the cut-off state. The magnetic resonance transmitter transmits the radio frequency signal to the coil along a direction of the third capacitor C3, the first diode D1, the second diode D2, and the fifth capacitor C5. Since the first inductor L1, the second inductor L2, and the fifth inductor L5 have a function of isolating an alternating current signal, the radio frequency signal may be avoided from escaping from the transmission path during transmission, and a link loss of the radio frequency signal is reduced. Moreover, since the third capacitor C3 can isolate a direct current, the direct current output from the first direct current power supply 21 cannot reach the magnetic resonance transmitter.

When the coil is required to send the collected magnetic resonance signal to the magnetic resonance receiver, the second direct current power supply 22 outputs the second turn-on signal. The third diode D3 is conducted along a direction of the fourth inductor L4, the third diode D3, and the fifth inductor L5. The fourth diode D4 is conducted along a direction of the fourth inductor L4, the fourth diode D4, and the third inductor L3. At this time, the transmission channel from the coil to the magnetic resonance receiver is opened. At the same time, the first direct current power supply 21 outputs the first cut-off signal, causing the first diode D1 and the second diode D2 to be in the cut-off state, i.e., the magnetic resonance transmitter to the coil is in the cut-off state. The magnetic resonance signal collected by the coil is transmitted to the magnetic resonance receiver along a direction of the fifth capacitor C5, the third diode D3, the fourth diode D4, and the fourth capacitor C4. Since the third inductor L3, the fourth inductor L4, and the fifth inductor L5 have the function of isolating an alternating current signal, the collected signal may be avoided from escaping from the transmission path during transmission, and the link loss of the collected signal is reduced. Moreover, since the fourth capacitor C4 can isolate a direct current, the direct current output from the second direct current power supply 22 cannot reach the magnetic resonance receiver.

By providing the third capacitor C3, the fourth capacitor C4, and the fifth capacitor C5 in the magnetic resonance circuit, the direct currents can be effectively avoided from passing through the magnetic resonance transmitter, the magnetic resonance receiver, and the coil, thereby protecting devices in the magnetic resonance system and improving the safety of the magnetic resonance system.

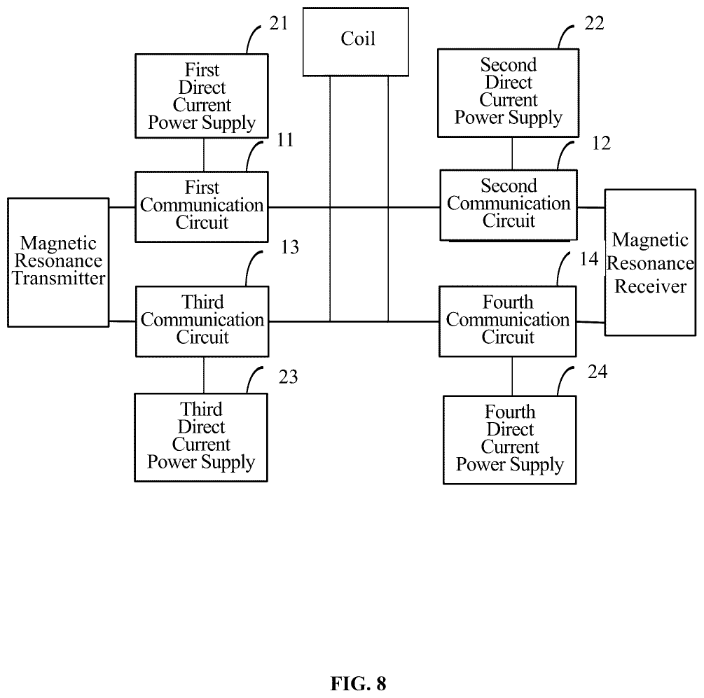

FIG. 8 is a schematic diagram illustrating a structure of a magnetic resonance circuit according to some embodiments of the present disclosure.

As shown in FIG. 8, in some embodiments, the communication circuit 10 may further include a third communication circuit 13 and a fourth communication circuit 14. The control circuit 20 may include a first control circuit and a second control circuit. The first communication circuit 11 and the third communication circuit 13 may be used to transmit the radio frequency signal transmitted by the magnetic resonance transmitter to the coil. The second communication circuit 12 and the fourth communication circuit 14 may be used to transmit the magnetic resonance signal collected by the coil to the magnetic resonance receiver. The first control circuit may be used to send a first control signal to the communication circuit 10 to control the first communication circuit 11 or the second communication circuit 12 to turn on or cut off. The second control circuit may be used to send a second control signal to the communication circuit 10 to control the third communication circuit 13 or the fourth communication circuit 14 to turn on or cut off.

In some embodiments, there may be two transmission channels included between the magnetic resonance transmitter and the coil, i.e., the first communication circuit 11 and the third communication circuit 13. There may also be two transmission channels between the coil and the magnetic resonance receiver, i.e., the second communication circuit 12 and the fourth communication circuit 14. In some embodiments, the first control circuit may be used to send the first control signal to the communication circuit 10 to control the first communication circuit 11 to be turned on or the second communication circuit 12 to be turned on or cut off. The second control circuit may be used to send the second control signal to the communication circuit 10 to control the third communication circuit 13 or the fourth communication circuit 14 to be turned on or cut off.

In some embodiments, the first control circuit may include the first direct current power supply 21 and the second direct current power supply 22. The second control circuit includes a third direct current power supply 23 and a fourth direct current power supply 24. Specifically, as shown in FIG. 8, the first communication circuit 11 and the third communication circuit 13 are respectively connected to the magnetic resonance transmitter and the coil. The second communication circuit 12 and the fourth communication circuit 14 are respectively connected to the coil and the magnetic resonance receiver. The first direct current power supply 21 is connected to the first communication circuit 11, the second direct current power supply 22 is connected to the second communication circuit 12, the third direct current power supply 23 is connected to the third communication circuit 13, and the fourth direct current power supply 24 is connected to the fourth communication circuit 14. The first direct current power supply 21, the second direct current power supply 22, the third direct current power supply 23, and the fourth direct current power supply 24 are respectively used to control the turn-on and cut-off of the first communication circuit 11, the second communication circuit 12, the third communication circuit 13, and the fourth communication circuit 14.

It should be noted that the structures and functions of the third communication circuit 13 and the fourth communication circuit 14 are similar to structures and functions of the first communication circuit 11 and the second communication circuit 12, and structures of the third direct current power supply 23 and the fourth direct current power supply 24 are similar to the structures and functions of the first direct current power supply 21 and the second direct current power supply 22, which may be found in the relevant descriptions of FIG. 2-FIG. 7 of the present disclosure, and will not be repeated herein.

In some embodiments, when the first direct current power supply 21 and the third direct current power supply 23 output the first turn-on signal and a third turn-on signal, the first communication circuit 11 and the third communication circuit 13 are conducted. When the second direct current power supply 22 and the fourth direct current power supply 24 output the second cut-off signal and the fourth cut-off signal, the second communication circuit 12 and the fourth communication circuit 14 are cut off. At this time, the magnetic resonance transmitter and the coil are in a dual-channel communication state, and the magnetic resonance transmitter transmits two radio frequency signals to the coil. In some embodiments, when the second direct current power supply 22 and the fourth direct current power supply 24 output the second turn-on signal and the fourth turn-on signal, the second communication circuit 12 and the fourth communication circuit 14 are conducted; and when the first direct current power supply 21 and the third direct current power supply 23 output the first cut-off signal and a third cut-off signal, the first communication circuit 11 and the third communication circuit 13 are cut off. At this time, the coil and the magnetic resonance transmitter are in the dual-channel communication state, and the coil collects the two magnetic resonance signals and transmits them to the magnetic resonance receiver.

In some embodiments, through the two transmission channels between the magnetic resonance transmitter and the coil and the two transmission channels between the coil and the magnetic resonance receiver, the magnetic resonance transmitter may respectively output two radio frequency signals with a phase difference (e.g., 90 degrees) to the magnetic resonance radio frequency power transceiver. In such cases, the magnetic resonance system includes a dual-channel transmission channel. In the magnetic resonance system, the magnetic resonance transmitter may adjust the phase and amplitude of the radio frequency signal through various types of related devices (e.g., a power divider, a phase shifter, an attenuator, etc.), thereby generating a multi-path parallel radio frequency signal with adjustable frequency, phase, and amplitude. In an ultra-high field magnetic resonance system, a multi-path radio frequency signal can solve problems such as emission field inhomogeneity and radio frequency energy absorption, and can improve a signal-to-noise ratio of a magnetic resonance image while speeding up the velocity of imaging. The dual-channel transmission channel of the embodiments of the present disclosure may respectively output two radio frequency signals with a phase difference to the magnetic resonance radio frequency power transceiver, thereby effectively improving the operating efficiency of transmission and reception of the radio frequency.

In some embodiments, the first communication circuit 11 may be used to transmit a first radio frequency signal generated by the magnetic resonance transmitter to the coil, the second communication circuit 12 may be used to transmit the first magnetic resonance signal emitted by the coil to the magnetic resonance receiver, the third communication circuit 13 may be used to transmit a second radio frequency signal generated by the magnetic resonance transmitter to the coil, and the fourth communication circuit 14 may be used to transmit a second magnetic resonance signal emitted by the coil to the magnetic resonance receiver. The phase difference between the first radio frequency signal and the second radio frequency signal is 90 degrees.

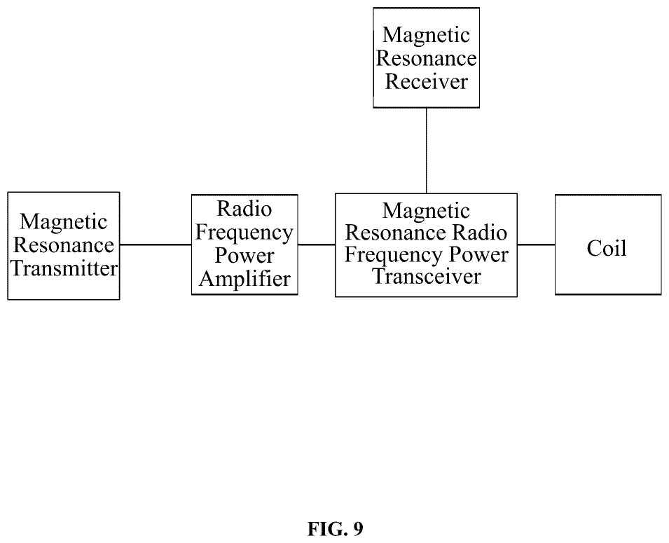

FIG. 9 is a schematic diagram illustrating a structure of a magnetic resonance system according to some embodiments of the present disclosure.

As shown in FIG. 9, in some embodiments, the magnetic resonance system may further include a radio frequency power amplifier for receiving the radio frequency signal output from the magnetic resonance transmitter and performing a power amplification. One end of the radio frequency power amplifier is connected to the magnetic resonance transmitter and another end is connected to the magnetic resonance radio frequency power transceiver.

In some embodiments, if the magnetic resonance radio frequency power transceiver is in the transmitting state, i.e., in the first operating mode, the magnetic resonance transmitter outputs the radio frequency signal to the radio frequency power amplifier, and the radio frequency power amplifier receives the radio frequency signal and performs the power amplification on the radio frequency signal, thereby avoiding a situation in which the detection results are not sufficiently accurate due to the power of the radio frequency signal being too low. If the power of the radio frequency signal is too low, the radio frequency signal may not be able to resonate with the nuclide and cannot generate the magnetic resonance signal. In some embodiments, in order to be suitable for the magnetic resonance environment in which the magnetic resonance system is applied and to support the transmission and power amplification of a plurality of radio frequency signals corresponding to different kinds of nuclides, the radio frequency power amplifier needs to have a large broadband power and bandwidth. Merely by way of example, the broadband power of the radio frequency power amplifier may be 1000 W and a bandwidth of the radio frequency power amplifier may be 30-405 MHz.

In some embodiments, the radio frequency power amplifier may also filter the radio frequency signal output from the magnetic resonance transmitter. The magnetic resonance transmitter may generate a plurality of radio frequency signals corresponding to different kinds of nuclides. If one of the nuclides (e.g., hydrogen nucleus) needs to be detected, the radio frequency power amplifier may amplify the radio frequency signal corresponding to the nuclide and transmit the radio frequency signal to the coil via the magnetic resonance radio frequency power transceiver. In some embodiments, the radio frequency power amplifier may be a single frequency point power amplifier, thereby amplifying the radio frequency signal at a particular frequency point and filtering out the radio frequency signals at other frequency points. In some embodiments, the radio frequency power amplifier may include a plurality of single frequency point power amplifiers, thereby performing the transmission and power amplification of a plurality of radio frequency signals corresponding to different kinds of nuclides with different frequencies.

In some embodiments, the radio frequency power amplifier may also not filter the radio frequency signal output from the magnetic resonance transmitter.

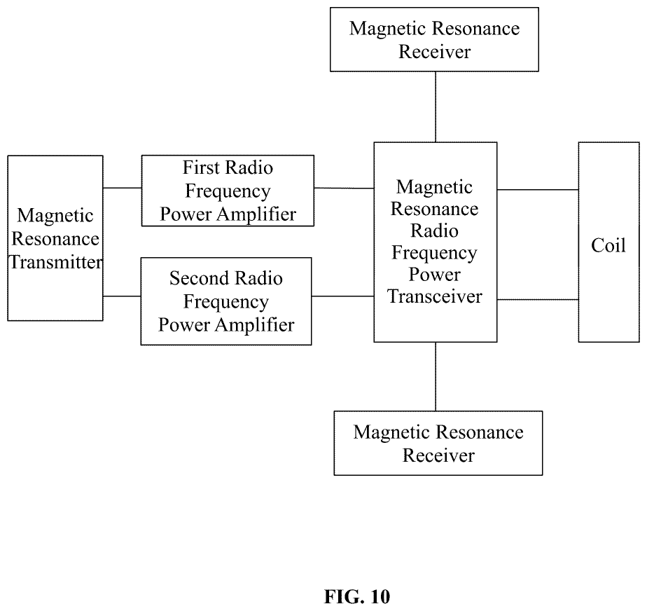

FIG. 10 is a schematic diagram illustrating a structure of a magnetic resonance system according to some embodiments of the present disclosure.

In some embodiments, if the magnetic resonance system includes a dual-channel transmission channel, the magnetic resonance transmitter may be used to output two radio frequency signals with a phase difference of 90 degrees to the magnetic resonance radio frequency power transceiver. Exemplarily, in the magnetic resonance system, there are dual channels included between the magnetic resonance transmitter and the coil, and between the coil and the magnetic resonance receiver. The magnetic resonance transmitter outputs two transmission signals with a phase difference of 90 degrees and transmits them to the magnetic resonance radio frequency power transceiver through the dual-channel between the magnetic resonance transmitter and the coil, and finally transmits them to the coil.

As shown in FIG. 10, the magnetic resonance system may include a first radio frequency power amplifier and a second radio frequency power amplifier. The first radio frequency power amplifier and the second radio frequency power amplifier are connected in parallel between the magnetic resonance transmitter and the magnetic resonance radio frequency power transceiver, i.e., one end of the first radio frequency power amplifier and one end of the second radio frequency power amplifier are jointly connected to the magnetic resonance transmitter, and another ends of them are jointly connected to the magnetic resonance radio frequency power transceiver.

In the first operating mode, the magnetic resonance transmitter transmits two radio frequency signals with a phase difference of 90 degrees to the first radio frequency power amplifier and the second radio frequency power amplifier, respectively. Two high-power radio frequency signals with the phase difference of 90 degrees are generated after power amplification, and they can be sent to the coil through the magnetic resonance radio frequency power transceiver. The coil further generates a magnetic field (e.g., a circularly polarized magnetic field generated through orthogonal excitation) to excite the nuclide to resonate and thereby generating the magnetic resonance signal. In the second operating mode, the coil collects the magnetic resonance signal and sends the magnetic resonance signal via the magnetic resonance radio frequency power transceiver to the magnetic resonance receiver for subsequent processing.

In some embodiments, the magnetic resonance radio frequency power transceiver may further include two radio frequency power switches to replace the communication circuit 10 and the control circuit 20. The two radio frequency power switches support the signal transmission of the plurality of radio frequency signals and may be used to determine the operating mode of the magnetic resonance radio frequency power transceiver.

Merely by way of example, the magnetic resonance radio frequency power transceiver may include two radio frequency power switches. Each of the two radio frequency power switches respectively controls one of the dual channels, thereby realizing the switching of the operating modes. Moreover, each of the two radio frequency power switches is capable of implementing a wide bandwidth transmission, i.e., supporting signal transmission of a plurality of frequencies. In some embodiments, each of the two radio frequency power switches may be an independent electronic component supporting a wide bandwidth range, or may be a combination of a plurality of electronic components corresponding to the plurality of frequencies. In some embodiments, to support the signal transmission of the plurality of radio frequency signals corresponding to different kinds of nuclides, i.e., to realize a wide bandwidth transmission, a frequency point supported by each of the two radio frequency power switches is within a range of 100-500 MHz, and a rated power of the radio frequency power switch may be 1,000 W. Merely by way of example, the frequency points supported by each of the two radio frequency power switches may include 105.92 MHz, 110.81 MHz, 376.84 MHz, or 400.55 MHz.

Specifically, the radio frequency power switches in the embodiments of the present disclosure enable the transmission and reception of the different kinds of nuclides (e.g., hydrogen (1H), the carbon (13C), the nitrogen (15N), the fluorine (19F), the sodium (23Na), the phosphorus (31P), and the xenon (129Xe)) in a high-field (e.g., magnetic field strength of 5T and above) magnetic resonance broadband without the need for switching, as well as without the need for switching the radio frequency power switches to match different frequencies.

The magnetic resonance radio frequency power transceiver in the embodiments of the present disclosure may include two radio frequency power switches. The radio frequency power switches support the transmission of signals with a plurality of frequencies, which can be used to determine the operating mode of the magnetic resonance radio frequency power transceiver. The broadband transmission is realized through the radio frequency power switches supporting multiple frequencies without the need to switch the radio frequency power switches to match different frequencies, which reduces the count of switches in the magnetic resonance system, thereby reducing the volume and the hardware cost of the magnetic resonance system.

The beneficial effects provided by the embodiments of the present disclosure include but are not limited to: (1) by the magnetic resonance radio frequency power transceiver and entering the first operating mode or the second operating mode according to the control signal, thereby realizing the switching between the transmission channel between the magnetic resonance transmitter and the coil and the transmission channel between the coil and the magnetic resonance receiver, there is no need to provide different transmission channels for excitations (or reception) of different nuclides, avoiding the use of too many hardware switches; (2) by adopting a magnetic resonance radio frequency power transceiver that supports broadband communication at a plurality of frequency points, the count of transmission channels between the magnetic resonance transmitter and the coil, and between the coil and the magnetic resonance receiver can be effectively reduced, thereby reducing the volume and the hardware cost of the magnetic resonance system and improving the operating efficiency of the transmission and reception of the radio frequency; (3) by respectively controlling the turn-on and cut-off of the first communication circuit and the second communication circuit using the first direct current power supply and the second direct current power supply, there is no need for a complex circuit control structure, the implementation process is simple, and the hardware cost of the control circuit is reduced; (4) by providing inductor elements to isolate radio frequency signals and collect signals during transmission, the losses of the radio frequency signals and the collected magnetic resonance signals during transmission are avoided to ensure the signal quality, thereby enhancing the detection accuracy of the magnetic resonance system; (5) the interferences from signals other than the control signals, the plurality of radio frequency signals, and the plurality of magnetic resonance signals are filtered out using the filter circuit composed of capacitive elements and inductor elements to filter out; (6) by providing the capacitive element in the magnetic resonance circuit, the direct currents are effectively prevented from passing through the magnetic resonance transmitter, the magnetic resonance receiver, and the coil, thereby protecting the devices in the magnetic resonance system and improving the safety of the magnetic resonance system; (7) the dual-channel transmission channel can respectively output two radio frequency signals with a phase difference to the magnetic resonance radio frequency power transceiver, which improves the operating efficiency of the transmission and reception of the radio frequency.

It should be noted that different embodiments may produce different beneficial effects, and in different embodiments, the potential beneficial effects may be any one or a combination of one or more of the above, or any other beneficial effect that may be obtained.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure.

Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

What is claimed is:

1. A magnetic resonance system, comprising:
a magnetic resonance transmitter configured to generate one or more radio frequency signals;
a magnetic resonance receiver configured to receive one or more magnetic resonance signals collected by one or more coils; and
a magnetic resonance radio frequency power transceiver, including a communication circuit and a control circuit, wherein
the communication circuit provides a first communication circuit and a second communication circuit, the first communication circuit is configured to transmit the one or more radio frequency signals to the one or more coils, and the second communication circuit is configured to transmit the one or more magnetic resonance signals to the magnetic resonance receiver; and
the control circuit is configured to send one or more control signals to the communication circuit to control the first communication circuit or the second communication circuit to turn on or cut off.

2. The system of claim 1, wherein the one or more radio frequency signals are a plurality of radio frequency signals corresponding to different kinds of nuclides, the one or more magnetic resonance signals are a plurality of magnetic resonance signals sent by the different kinds of nuclides, and the second communication circuit is configured to transmit the plurality of magnetic resonance signals to the magnetic resonance receiver, respectively.

3. The system of claim 2, wherein the one or more coils are provided inside or connected to the magnetic resonance system and configured to generate a magnetic field that excites resonance of the different kinds of nuclides and/or to collect the plurality of magnetic resonance signals sent by the different kinds of nuclides.

4. The system of claim 3, wherein the control circuit includes a power supply, and the power supply is configured to output a turn-on signal and/or a cut-off signal to control the first communication circuit and the second communication circuit to turn on and/or cut off.

5. The system of claim 4, wherein the power supply includes a first direct current power supply and a second direct current power supply, the first direct current power supply is connected to the first communication circuit, and the second direct current power supply is connected to the second communication circuit.

6. The system of claim 5, wherein
the first communication circuit includes a first diode and a second diode, the first diode is connected to the magnetic resonance transmitter, and one end of the second diode is connected to the first diode and another end is connected to the one or more coils; and
the second communication circuit includes a third diode and a fourth diode, the third diode is connected to the one or more coils, and one end of the fourth diode is connected to the third diode and another end of the fourth diode is connected to the magnetic resonance receiver.

7. The system of claim 6, wherein the system further comprises a first inductor, a third inductor, and a fifth inductor, wherein
the first inductor is connected between the magnetic resonance transmitter and a ground end,
the third inductor is connected between the magnetic resonance receiver and the ground end, and
the fifth inductor is connected between the one or more coils and the ground end.

8. The system of claim 7, further comprising a second inductor and a fourth inductor, wherein
the second inductor is connected between the first direct current power supply and the first communication circuit, and
the fourth inductor is connected between the second direct current power supply and the second communication circuit.

9. The system of claim 7, further comprising a capacitive element, wherein the capacitive element forms a filter circuit with the second inductor and the fourth inductor, the filter circuit is configured to filter out interferences from other signals other than the one or more control signals, the plurality of radio frequency signals, and the plurality of magnetic resonance signals.

10. The system of claim 8, wherein the capacitive element includes a first capacitor and a second capacitor, the first capacitor is connected to the second inductor, and the second capacitor is connected to the fourth inductor, wherein the first capacitor and the second inductor form a first filter circuit, and the second capacitor and the second capacitor form a second filter circuit.

11. The system of claim 5, further comprising a third capacitor, a fourth capacitor, and a fifth capacitor, wherein
the third capacitor is connected between the magnetic resonance transmitter and the first communication circuit, and the third capacitor is configured to isolate a first direct current output from the first direct current power supply to avoid the first direct current passing through the magnetic resonance transmitter when the first communication circuit is turned on;
the fourth capacitor is connected between the magnetic resonance receiver and the second communication circuit, and the fourth capacitor is configured to isolate a second direct current output from the second direct current power supply to avoid the second direct current passing through the magnetic resonance receiver when the second communication circuit is turned on; and
the fifth capacitor is connected between the one or more coils and the communication circuit, and the fifth capacitor is configured to isolate the first direct current and the second direct current to avoid the first direct current and the second direct current passing through the one or more coils.

12. The system of claim 3, further comprising a radio frequency power amplifier, wherein the radio frequency power amplifier is configured to receive the plurality of radio frequency signals and perform a power amplification.

13. The system of claim 12, wherein a broadband power of the radio frequency power amplifier is 1000 W and a bandwidth of the radio frequency power amplifier is within a range of 30 to 405 MHz.

14. The system of claim 2, wherein the communication circuit further includes a third communication circuit and a fourth communication circuit, and the magnetic resonance transmitter is further configured to output two radio frequency signals with a phase difference of 90 degrees to the magnetic resonance radio frequency power transceiver.

15. The system of claim 14, wherein.
the first communication circuit is configured to transmit a first radio frequency signal generated by the magnetic resonance transmitter to the one or more coils,
the second communication circuit is configured to transmit a first magnetic resonance signal sent from the one or more coils to the magnetic resonance receiver,
the third communication circuit is configured to transmit a second radio frequency signal generated by the magnetic resonance transmitter to the one or more coils, and
the fourth communication circuit is configured to transmit a second magnetic resonance signal sent from the one or more coils to the magnetic resonance receiver,
wherein a phase difference between the first radio frequency signal and the second radio frequency signal is 90 degrees.

16. The system of claim 14, wherein the communication circuit includes two radio frequency power switches, the two radio frequency power switches support signal transmission at multiple frequencies and are configured to control an operating mode of the magnetic resonance radio frequency power transceiver.

17. The system of claim 16, wherein a frequency point supported by each of the two radio frequency power switches is within a range of 100-500 MHz, and a power of the radio frequency power switch is 1000W.

18. The system of claim 3, wherein the one or more coils includes multiple groups of coils, each group of coils respectively corresponds to the one or more radio frequency signals or the one or more magnetic resonance signals of the different kind of nuclide.

19. A magnetic resonance circuit applied to a magnetic resonance system, wherein the magnetic resonance system further comprises a magnetic resonance transmitter and a magnetic resonance receiver, and the magnetic resonance circuit includes a communication circuit and a control circuit, wherein:
the communication circuit provides a first communication circuit and a second communication circuit, the first communication circuit is configured to transmit one or more radio frequency signals generated by the magnetic resonance transmitter to one or more coils, and the second communication circuit is configured to transmit one or more magnetic resonance signals collected by the one or more coils to the magnetic resonance receiver; and
the control circuit is configured to send one or more control signals to the communication circuit to control the first communication circuit or the second communication circuit to turn on or cut off.

20. The circuit of claim 19, wherein
the first communication circuit is configured to transmit a plurality of radio frequency signals corresponding to different kinds of nuclides generated by the magnetic resonance transmitter to the one or more coils, and
the second communication circuit is configured to transmit a plurality of magnetic resonance signals sent from different kinds of nuclides collected by the one or more coils to the magnetic resonance receiver, respectively.
